(19) United States
(12) Patent Application Publication
(10) Pub. No.: US 2008/0249417 A1
(43) Pub. Date: Oct. 9, 2008

(54) Non-Linear Ultrasonic Diagnostic Imaging Using Intermodulation Product Signals

(75) Inventors: Michalakis Averkiou, Kirkland, WA (US); Seth Jensen, Bothell, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/570,607

(22) PCT Filed: Jun. 22, 2005
(86) PCT No.: PCT/IB05/52056

§ 371 Date: Dec. 14, 2006

Related U.S. Application Data
(60) Provisional application No. 60/584,403, filed on Jun. 30, 2004.

(51) Int. Cl. A61B 8/00

(52) U.S. Cl. 600/459

(57) ABSTRACT

An ultrasonic imaging system transmits waveforms containing first and second major frequency components which are intermodulated by passage through a nonlinear medium or interaction with a contrast agent microbubble to produce a difference frequency component. In an illustrated embodiment the second major frequency is twice the frequency of the first major frequency, resulting in a difference frequency signal at the first major frequency. Two differently modulated transmit waveforms are transmitted and the difference frequency component is separated by pulse inversion.

This invention relates to medical diagnostic imaging systems and, in particular, to ultrasonic diagnostic imaging systems in which nonlinear intermodulation products of transmitted signals are used for imaging.

Imaging with nonlinear signals presently finds two major applications in diagnostic ultrasound. One is tissue harmonic imaging in which a linear (generally sinusoidal) transmit waveform is allowed to undergo natural distortion as it passes through the body. The distortion gives rise to the development of nonlinear harmonic components of which the most significant is usually at the second harmonic of the fundamental transmit frequency. The received echoes are filtered to separate the nonlinear components from the linear components. A preferred separation technique is known as pulse inversion as described in U.S. Pat. No. 5,951,478 (Hwang et al.) Images produced from the nonlinear components are desirable for their low level of clutter due to multipath scattering.

The second significant application of nonlinear imaging is the imaging of ultrasonic contrast agents. The microbubbles of contrast agents can be designed to oscillate nonlinearly or break up when insonified by ultrasound. This oscillation or destruction will cause the echoes returned from the microbubbles to be rich in nonlinear components. The echoes are received and processed in a similar manner as tissue harmonic signals to separate the nonlinear components of the microbubble echoes. Images produced with these echoes can sharply segment the blood flow and vasculature containing the contrast agent.

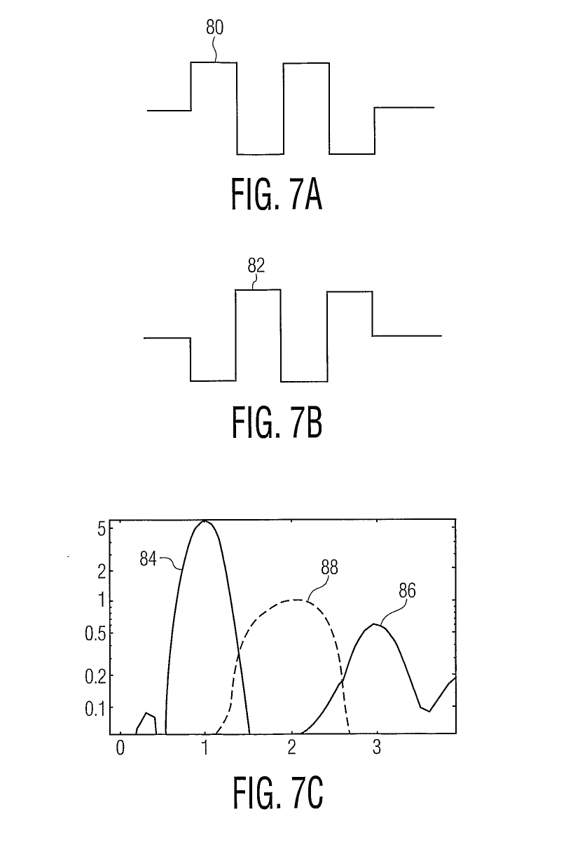

U.S. Pat. No. 6,440,075 (Averkiou) describes a nonlinear imaging technique which enhances the production of nonlinear signal components. This is done by transmitting a waveform with two major frequencies. As the waveform passes through tissue or encounters a microbubble nonlinear components of each transmit frequency will be developed as described above. In addition, the two transmit frequency components will intermodulate, thereby developing nonlinear sum and difference frequency components. Both types of nonlinear signals are received and used to form images which are enhanced by the use of two nonlinearity mechanisms. This patent gives examples of several ways in which sum and difference frequencies can be formed and located, such as by using the sides of the transducer passband for the major transmit frequencies and the center for difference and harmonic frequencies. FIG. 7 of the '075 patent gives an example of the transmission of frequencies f₁ and f₂ at the sides of the transducer passband and the reception of echo components f₁−f₂ and 2f₂ in the center of the passband. The illustrated transmission techniques may also be advantageously produced from digitally stored transmit waveforms.

For imaging at greater depths in the body, which is often necessary for deep abdominal imaging such as imaging the liver, lower frequencies are required to counter the effects of depth-dependent frequency attenuation. As the examples in the '075 patent illustrate, the intermodulation products are often at the center of the passband or higher and can therefore suffer from substantial attenuation in deeper depth imaging. This attenuation can reduce the signal-to-noise characteristic of the received echoes and hence the diagnostic quality of the images. It is therefore desirable to be able to employ intermodulation nonlinear imaging in a way which will produce highly diagnostic images when imaging at greater depths in the body.

In accordance with the principles of the present invention, a method and apparatus for nonlinear imaging with intermodulation products at greater depths are described. The transmit waveform contains two major frequency components, one of which is twice the frequency of the other. The transmit waveform is transmitted twice, each time with a different transmit modulation. The received echoes from the two transmissions are combined to separate nonlinear difference frequency components of the two major frequency components by pulse inversion. The difference frequency components are located at the lower of the two major frequency components and hence are less susceptible to the effects of depth-dependent attenuation.

In the drawings:

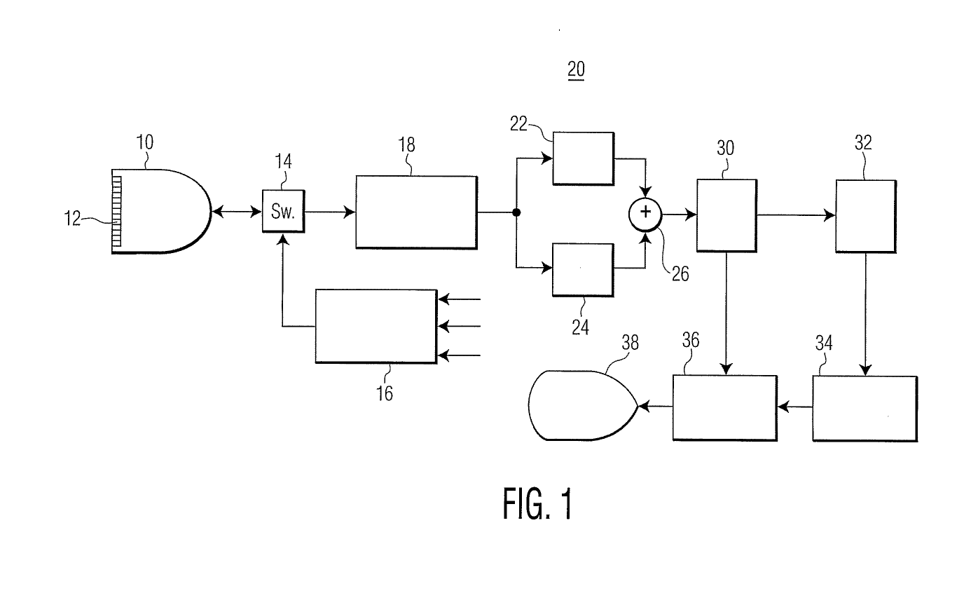

FIG. 1 illustrates in block diagram form an ultrasonic diagnostic imaging system constructed in accordance with the principles of the present invention.

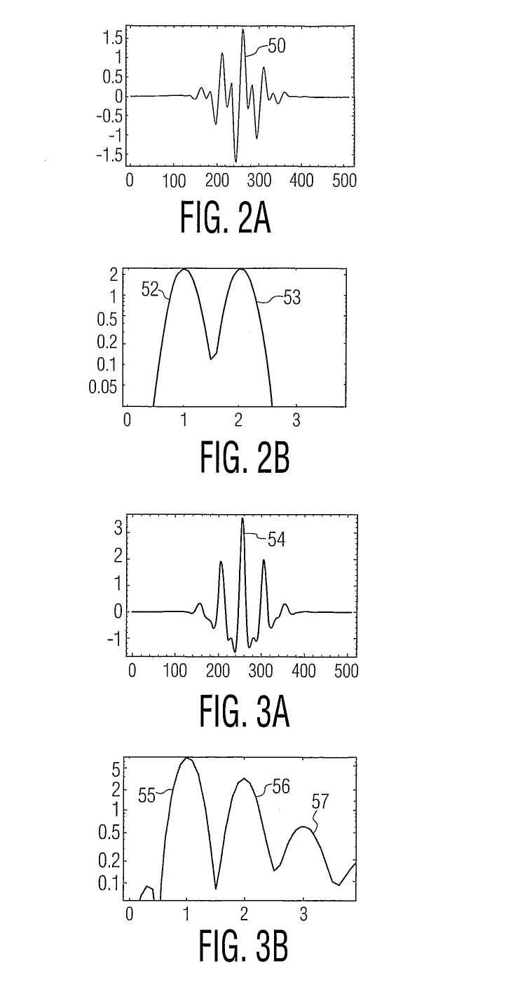

FIGS. 2A-5B illustrate waveforms used to produce nonlinear echo signal components in accordance with the principles of the present invention.

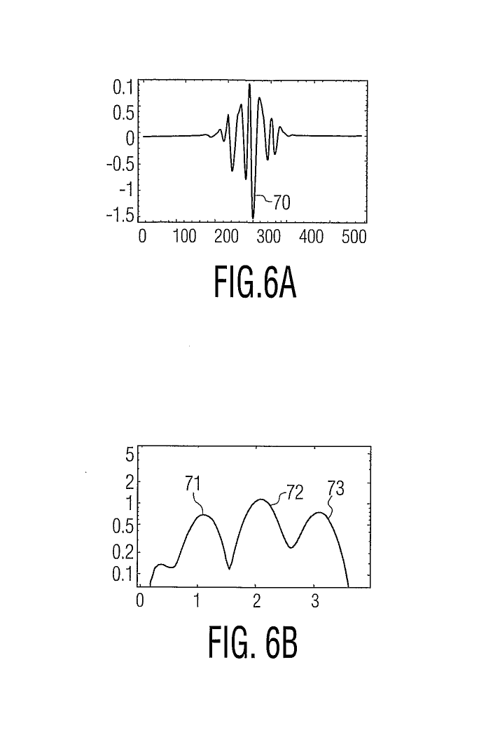

FIGS. 6A and 6B illustrate the result of pulse inversion separation using the echo signals of FIGS. 3A and 5A.

FIGS. 7A and 7B illustrate two differently modulated transmit square waves in accordance with another embodiment of the present invention.

FIG. 7C illustrates the spectrum of the transmit square waves of FIGS. 7A and 7B and the nonlinear components of the received echo signals.

Referring first to FIG. 1, an ultrasonic diagnostic imaging system constructed in accordance with the principles of the present invention is shown. The ultrasound system of FIG. 1 utilizes a transmitter 16 which transmits multifrequency beams for the nonlinear generation of difference frequency signals within the subject being imaged. The transmitter is coupled by a transmit/receive switch 14 to the elements of an array transducer 12 of a scanhead 10. The transmitter is responsive to a number of control parameters which determine the characteristics of the transmit beams, as shown in the drawing. The two major frequencies f₁ and f₂ of the multifrequency beam are controlled, which determine the frequency at which difference (f₁−f₂) frequency components will fall. Also controlled are the amplitudes or intensities a and b of the two transmitted frequency components, causing the transmit beam to be of the form (bsin(2πf₁t)+asin(2πf₂t)). The received difference signal component (f₁−f₂) will have an amplitude c which is not a linear product of the a and b intensities, however, as the difference signal results from nonlinear effects.

In FIG. 1, the transducer array 12 receives echoes from the body containing the difference frequency components which are within the transducer passband. These echo signals are coupled by the switch 14 to a beamformer 18 which appropriately delays echo signals from the different elements then combines them to form a sequence of difference signals along the beam from shallow to deeper depths. Preferably the beamformer is a digital beamformer operating on digitized echo signals to produce a sequence of discrete coherent digital echo signals from a near to a far depth of field. The beamformer may be a multiline beamformer which produces two or more sequences of echo signals along multiple spatially distinct receive scanlines in response to a single transmit beam. The beamformed echo signals are coupled to a nonlinear signal separator 20. The separator 20 may be a bandpass filter which passes a sum or difference passband 66,76 to the relative exclusion (attenuation) of the transmit bands 62,64 or 72,74. In the illustrated embodiment the separator 20 is a pulse inversion processor which separates the nonlinear signals including the difference frequency components by the pulse inversion technique. Since the difference frequency signals are developed by nonlinear effects, they may advantageously be separated by pulse inversion processing. For pulse inversion the transmitter has another variable transmit parameter which is the phase, polarity or amplitude of the transmit pulse as shown in the drawing. The ultrasound system transmits two or more beams of different transmit polarities, amplitudes and/or phases. For the illustrated two pulse embodiment, the scanline echoes received in response to the first transmit pulse are stored in a Line1 buffer 22. The scanline echoes received in response to the second transmit pulse are stored in a Line2 buffer 24 and then combined with spatially corresponding echoes in the Line1 buffer by a summer 26. Alternatively, the second scanline of echoes may be directly combined with the stored echoes of the first scanline without buffering. As a result of the different phases or polarities of the transmit pulses, the out of phase fundamental (linear) echo components will cancel and the nonlinear difference frequency components, being in phase, will combine to reinforce each other, producing enhanced and isolated nonlinear difference frequency signals. The difference frequency signals may be further filtered by a filter 30 to remove undesired signals such as those resulting from operations such as decimation. The signals are then detected by a detector 32, which may be an amplitude or phase detector. The echo signals are then processed by a signal processor 34 for subsequent grayscale, Doppler or other ultrasound display, then further processed by an image processor 36 for the formation of a two dimensional, three dimensional, spectral, parametric, or other display. The resultant display signals are displayed on a display 38.

In accordance with the principles of the present invention the transmitter transmits waveforms with two major transmit frequencies, f₁ and f₂, where f₂=2f₁. These two transmit frequencies will be intermodulated within the body due to nonlinear effects such as the passage of the waveform through tissue or reflection by a nonlinear contrast agent microbubble. This intermodulation produces components at the sum and difference frequencies of the two major frequencies. As a result of the selected major frequencies, the difference frequency f₂−f₁=f₁, which comprise nonlinear signal components at the lower transmit frequency. Since the lower transmit frequency will exhibit the greatest depth of penetration, nonlinear signal components will be returned from the greatest depth at which the lowest frequency f₁ can be received. Thus, imaging at greater depths is facilitated.

An example of this process is illustrated by FIGS. 2A through 6B. FIG. 2A is a graphical time domain drawing of a first transmit waveform 50 which exhibits a first modulation characteristic which in this example is a specific phase characteristic. The abscissa of the graph is time and the ordinate is amplitude. The transmit waveform 50 has two major frequency components which are shown in FIG. 2B. This graphical drawing shows the frequency spectrum of the transmit waveform 50. The abscissa of the graph can be considered a frequency scale in MHz or order of harmonic and the ordinate is amplitude. The spectrum shows that the first transmit waveform has a first major frequency component 52 around 1 MHz and a second major frequency component 53 around 2 MHz. The second major frequency component 53 is seen to be twice the value of the first major frequency component. Alternatively the spectrum can be viewed as having two major fundamental frequency components of which the higher frequency component is at the second harmonic frequency of the lower frequency component.

When the first transmit waveform is directed to a nonlinear medium or target an echo 54 is returned and received by the transducer 12 as shown in FIG. 3A. This echo has a spectral response as shown in FIG. 3B. This spectrum includes fundamental frequency components 55, 56, and 57. For ease of explanation the response characteristic 55 will be referred to as the fundamental response, the characteristic 56 as the second harmonic response, and the response characteristic 57 as the third harmonic response. The fundamental component 55 includes the linear response from the transmit component 52 and also the nonlinear response from the intermodulation product of the transmit frequencies. In this case the intermodulation product is the difference frequency f₁−f₂, which in this example where f₂=2f₁ is equal to f₁. The second harmonic component 56 is the linear response from transmit component 53 and the second harmonic a nonlinear response of transmit component 52. The third harmonic component 57 is solely a nonlinear response. This component includes the third harmonic component of transmit frequency component 52 and the sum of intermodulation frequency f₁+f₂ which in this case is equal to 3f₁. The echo signal 54 is beamformed and stored in the Line1 buffer 22.

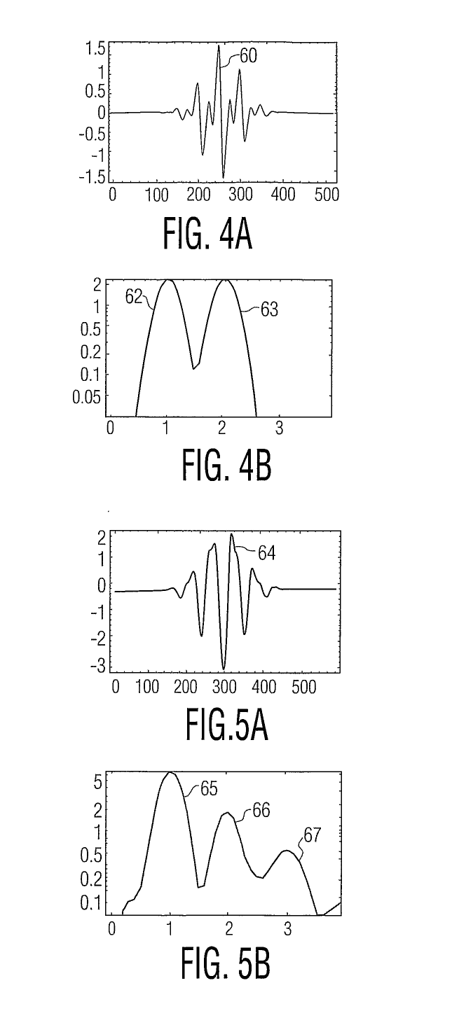

A second transmit waveform 60 is transmitted to the same target or medium as the first waveform 50 as shown in FIG. 4A. This second transmit waveform is differently modulated from the first transmit waveform, in this example by a different phase characteristic. The spectral characteristics 62 of the second transmit waveform are shown in FIG. 4B, which are seen to be the same as that of the first transmit waveform and exhibiting the first and second major frequency components. The echo 64 received from the medium or target in response to the second transmit waveform is shown in FIG. 5B and is seen to differ from the echo 54 from the first transmit waveform by reason of the different phase modulation of the waveform. The echo signal 64 has substantially the same spectral characteristics as those of the echo 54, as can be seen by the spectral response curves 65, 66 and 67 in FIG. 5B. The echo from the second transmit waveform includes fundamental components of the first and second major frequency components of the transmit waveform, a third harmonic of the first (lower) major frequency component, a nonlinear (second) harmonic of the first and second major frequency components, and the difference signal intermodulation product of the two major frequency components at 1 MHz. The echo signal 64 is beamformed and stored in the Line2 buffer 24.

The nonlinear components of the echo signals are separated by pulse inversion by adding the two stored echoes with the summer 26. The combining of the two signals causes the linear components to cancel each other by reason of the different modulation of the transmit waveforms, and allows the nonlinear components of the two echoes to reinforce each other. The result of this combining for this example is the signal 70 shown in FIG. 6A. The frequency spectrum of this signal is shown in FIG. 6B and has three distinct components 71, 72 and 73. This spectrum is seen to include nonlinear components 2f₁ and 3f₁ of the first major frequency component f₁ at the second and third harmonic frequencies of the f₁ frequency. The spectrum also has a nonlinear component at the fundamental frequency of the f₁ component, which is the difference frequency of the first and second major frequency components and another contribution at 3f₁. which is the sum frequency of the first and second major frequency components. When the transmit waveforms are transmitted to and echoes received from substantial depths of field, the received echoes can be expected to be significantly affected by depth-dependent frequency attenuation. This will cause significant attenuation of the higher second and third harmonic frequencies, resulting in faint or noisy second harmonic images. However the difference frequency component is at the same low frequency f₁ as the first frequency component because of the use of f₂=2f₁. That is, 2f₁−f₁=f₁. Since this component is a nonlinear intermodulation product which develops within the subject it will not suffer from the clutter effects of the fundamental (linear) f₁ transmit signal itself. The frequency attenuation of the difference frequency component will be no greater than that of the f₁ frequency, enabling the production of more diagnostically effective images from greater depths of field as nonlinear images can be formed with components from f₁, 2f₁, and 3f₁ frequencies. Additionally the different frequency components f₁, 2f₁ and 3f₁ can be combined to reduce speckle artifacts in the image as described in U.S. Patent application Ser. No. 60/527,538.

When the transmit waves are modulated from pulse to pulse in both phase and amplitude, the following spectrum will result. The first harmonic frequency range will include the nonlinear fundamental components of transmit frequencies 52 and 62 plus the difference frequency of 53−52 and 63−62. The second harmonic frequency range will include the nonlinear fundamental components of frequency 53 and the second harmonic of frequency 52. The third harmonic response will include the third harmonic of frequency 52 and the sum frequency of frequencies 52 and 53.

In accordance with a further aspect of the present invention, a transmit waveform with first and second major frequency components may be produced by a square waveform. FIGS. 7A and 7B illustrate first and second transmit waveforms which are differently modulated square waveforms 80 and 82. These waveforms are seen to be 180° out of phase with each other so as to produce echoes from which nonlinear components may be separated by the pulse inversion process. Square waveforms can be produced by inexpensive switching transmitters in which the output is produced by switching between different voltage rails. Such transmitters are more inexpensive to manufacture than transmitters which perform digital to analog conversion of digitally stored waveforms, which can produce exactly tailored transmit signals of specific wave shapes. This embodiment thus lends itself well to use in inexpensive ultrasound systems with simple switching transmitters.

The sharp switching of the squarewave signals cause the signals to be rich in harmonic frequency components. A square wave will produce a transmit signal with major frequency components at odd harmonic frequencies. FIG. 7C shows the frequency spectrum of a squarewave signal in the solid lines, which is seen to have a first major frequency component 84 at the fundamental (1^{st} harmonic) frequency f₁ and a second major frequency component 86 at the third harmonic frequency 3f₁, leaving the intermediate second harmonic frequency substantially free of transmit signal frequencies. The intermodulation of the first and second major frequency components 84 and 86 caused by the nonlinear medium or target, will create difference frequency components of 3f₁−f₁=2f₁ at the intermediate second harmonic frequency in the returning echo signal as indicated by the dashed passband 88. Passband 88 will also include second harmonics of the frequencies in passband 84. The received difference signals can be separated by bandpass filtering with a filter exhibiting the passband 88 or by pulse inversion separation which will further attenuate the received linear signal components. The received and separated nonlinear echo signals will thus be substantially uncontaminated by clutter and other components of the transmitted signals.

In summary, the passband 88 includes the second harmonic (2f₁) of the transmitted frequency components in passband 84 and the difference frequencies of the components 3f₁-f₁ in bands 84 and 86. When both phase (or polarity) and amplitude modulation are employed, the received components include the nonlinear fundamental frequency components of frequencies in transmit band 84; the second harmonic (2f₁) and difference frequency components (3f₁-f₁) in the intermediate band 88; and third harmonic (3f₁) components in the higher passband 86.

What is claimed is:

1. An ultrasonic diagnostic imaging system for nonlinear imaging with intermodulation product signals comprising:
an array transducer which acts to transmit ultrasonic waves and receive echo signals in response to the waves;
a transmitter, coupled to the array transducer, which causes the array transducer to transmit differently modulated first and second waves to a target location, each wave including first and second major frequency components, wherein the second major frequency component is twice the frequency of the first major frequency component;
a beamformer coupled to the array transducer which forms coherent echo signals in response to the transmitted waves, including a difference frequency signal of the first and second major frequency components;
a pulse inversion processor, coupled to the beamformer, which acts to separate the difference frequency signal of the first and second major frequency components to the relative exclusion of linear signal components of the first major frequency component; and
a display, coupled to the pulse inversion processor, for displaying an image formed from the difference frequency signal.

2. The ultrasonic diagnostic imaging system of claim 1, wherein the beamformer further comprises means for forming coherent echo signals in response to the transmitted waves, including a difference frequency signal of the first and second major frequency components at the frequency of the first major frequency component.

3. The ultrasonic diagnostic imaging system of claim 1, wherein the pulse inversion processor further comprises means for separating a nonlinear difference frequency signal of the first and second major frequency components to the relative exclusion of linear signal components of the first major frequency component.

4. The ultrasonic diagnostic imaging system of claim 1, wherein the transmitter further comprises means for causing the array transducer to transmit first and second waves to a target location which are differently modulated in amplitude.

5. The ultrasonic diagnostic imaging system of claim 1, wherein the transmitter further comprises means for causing the array transducer to transmit first and second waves to a target location which are differently modulated in at least one of phase or frequency.

6. The ultrasonic diagnostic imaging system of claim 1, wherein the transducer further comprises an array transducer which acts to transmit ultrasonic waves and receive echo signals in response to the waves from a depth of field from which higher frequency signals exhibit significant depth dependent frequency attenuation.

7. The ultrasonic diagnostic imaging system of claim 1, wherein the array transducer further comprises means for receiving echoes including difference frequency components formed by the intermodulation of the first and second major frequency components by a nonlinear target or medium.

8. The ultrasonic diagnostic imaging system of claim 7, wherein the nonlinear target comprises a contrast agent microbubble.

9. The ultrasonic diagnostic imaging system of claim 7, wherein the nonlinear medium comprises body tissue.

10. An ultrasonic diagnostic imaging system for nonlinear imaging with intermodulation product signals comprising:
an array transducer which acts to transmit ultrasonic waves and receive echo signals in response to the waves;
a transmitter, coupled to the array transducer, which causes the array transducer to transmit square waves to a target location, each square wave transmitting first and second major frequency components, wherein the second major frequency component is three times the frequency of the first major frequency component;
a beamformer coupled to the array transducer which forms coherent echo signals in response to the transmitted square waves, including a difference frequency signal of the first and second major frequency components;
a signal separation circuit, coupled to the beamformer, which acts to separate the difference frequency signal of the first and second major frequency components to the relative exclusion of linear signal components of the major frequency components; and
a display, coupled to the pulse inversion processor, for displaying an image formed from the difference frequency signal.

11. The ultrasonic diagnostic imaging system of claim 10, wherein the transmitter further comprises means for causing the array transducer to transmit differently modulated square waves to a target location, each square wave transmitting odd harmonics of a fundamental frequency.

12. The ultrasonic diagnostic imaging system of claim 11, wherein the transmitter further comprises means for causing the array transducer to transmit differently modulated square waves to a target location, each square wave transmitting the first and third harmonic frequencies of a fundamental frequency and a relative absence of signal content at the second harmonic frequency of the fundamental frequency.

13. The ultrasonic diagnostic imaging system of claim 12, wherein the beamformer further comprises means for forming coherent echo signals in response to the transmitted square waves, including a difference frequency signal of the first and second major frequency components located at the second harmonic frequency of the fundamental frequency.

14. The ultrasonic diagnostic imaging system of claim 12, wherein the beamformer further comprises means for forming coherent echo signals in response to the transmitted square waves, including an intermodulation product of the first and second major frequency components.

15. The ultrasonic diagnostic imaging system of claim 14, wherein the beamformer further comprises means for forming coherent echo signals in response to the transmitted square waves, including an intermodulation product of the first and second major frequency components, wherein the intermodulation product is located at the second harmonic frequency of the fundamental frequency.

16. The ultrasonic diagnostic imaging system of claim 10, wherein the transmitter further comprises a switching transmitter which acts to switch an output waveform between discrete voltage levels.

17. The ultrasonic diagnostic imaging system of claim 10, wherein the signal separator circuit further comprises a bandpass filter.

18. The ultrasonic diagnostic imaging system of claim 10, wherein the transmitter further comprises means for causing the array transducer to transmit first and second differently modulated square waves to a target location;
wherein the beamformer further comprises means for forming coherent echo signals in response to the first and second differently modulated square waves; and
wherein the signal separation circuit further comprises a pulse inversion circuit responsive to coherent echo signals received in response to the first and second differently modulated square waves.

19. The ultrasonic diagnostic imaging system of claim 18, wherein the transmitter further comprises means for causing the array transducer to transmit first and second square waves which are differently modulated in at least one of amplitude, phase or polarity.
